(19) United States
(12) Patent Application Publication
(10) Pub. No.: US 2024/0197658 A1
(43) Pub. Date: Jun. 20, 2024

(54) COMPOSITION FOR INHIBITING ANTI-CANCER DRUG RESISTANCE

(71) Applicant: AJOU UNIVERSITY INDUSTRY-ACADEMIC COOPERATION FOUNDATION, Suwon-si (KR)

(72) Inventors: Hoon HUR, Seongnam-si (KR); In-Hye Ham, Suwon-si (KR)

(73) Assignee: AJOU UNIVERSITY INDUSTRY-ACADEMIC COOPERATION FOUNDATION, Suwon-si (KR)

(21) Appl. No.: 18/531,132

(22) Filed: Dec. 6, 2023

(30) Foreign Application Priority Data
Dec. 9, 2022 (KR) 10-2022-0171413

(51) Int. Cl. A61K 31/167; A61P 35/00

(52) U.S. Cl. CPC A61K 31/167; A61P 35/00; A61K 2300/00

(57) ABSTRACT

Provided is a composition for inhibiting anti-cancer drug resistance comprising a compound represented by Chemical Formula 1 below as an active ingredient:

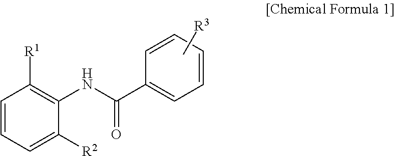

(wherein,
R¹ and R² are each independently hydrogen, substitutable linear or branched C₁-C₁₂ alkyl, substitutable C₃-C₁₂ cycloalkyl, or substitutable C₆-C₁₈ aryl,
R³ is carboxyl, hydroxyl, or acetyl, and
the substitution is performed by oxygen, nitrogen, sulfur, linear or branched C₁-C₆ alkyl, C₆-C₂₀ aryl, halogen, or a combination thereof).

### CROSS-REFERENCE TO RELATED APPLICATIONS

This application claims the priority of Korean Patent Application No. 10-2022-0171413 filed on Dec. 9, 2022, in the Korean Intellectual Property Office, the disclosure of which is incorporated herein by reference.

### BACKGROUND

### Field

The present disclosure relates to a composition for inhibiting anti-cancer drug resistance.

### Description of the Related Art

Cancer is a disease having mortality rate that continues to increase every year due to changes in the environment, aging population, and changes in lifestyle. Cancer is one of the top five causes of death worldwide and a disease that occupies the first cause of death in Korea. Cancer is treated with surgery, radiation therapy, and drug therapy. In the case of drug therapy, starting with first-generation chemotherapy, second-generation targeted drugs therapy was developed and are currently the most widely used, but are facing a major problem called anti-cancer drug resistance. To overcome the anti-cancer drug resistance, third-generation immunotherapy has been developed and side effects have been reduced, but there are still limitations in inhibiting anti-cancer drug resistance, and research on anti-cancer drug resistance is continuously ongoing.

Recently, in relation to anti-cancer drug resistance, the existence and roles of not only cancer cells but also immune cells, vascular cells, and cancer-associated fibroblasts constituted around cancer cells have been found. Among these, there are reports that cytokines or chemokines secreted from cancer-associated fibroblasts are related to increased drug resistance and poor prognosis in gastric cancer. Accordingly, studies of analyzing the impact of cancer-associated fibroblasts on cancer treatment have been conducted.

However, detailed contents on substances, methods, mechanisms of action, etc. for inhibiting anti-cancer drug resistance of cancer cells induced by cancer-associated fibroblasts are lacking to date.

Korean Patent Registration No. 10-1921676, which is the background art of the present disclosure, relates to a composition for inhibiting anti-cancer drug resistance containing a TESK1 inhibitor and a screening method for a TESK1 inhibitor. Korean Patent Registration No. 10-1921676 discloses a composition for inhibiting anti-cancer drug resistance by inhibiting actin remodeling. There is not disclosed the contents for inhibiting anti-cancer drug resistance of cancer cells induced by cancer-associated fibroblasts.

### SUMMARY

An object to be achieved by the present disclosure is to provide a composition for inhibiting anti-cancer drug resistance.

Another object to be achieved by the present disclosure is to provide a pharmaceutical composition for preventing or treating cancer including the composition for inhibiting anti-cancer drug resistance.

Objects of the present disclosure are not limited to the above-mentioned objects, and other objects, which are not mentioned above, can be clearly understood by those skilled in the art from the following descriptions.

According to an aspect of the present disclosure, there is provided a composition for inhibiting anti-cancer drug resistance including a compound represented by Chemical Formula 1 below as an active ingredient:

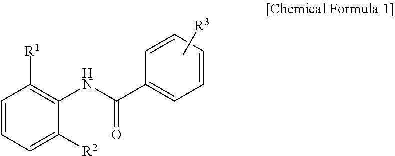

(wherein,
R¹ and R² are each independently hydrogen, substitutable linear or branched C₁-C₁₂ alkyl, substitutable C₃-C₁₂ cycloalkyl, or substitutable C₆-C₁₈ aryl,
R³ is carboxyl, hydroxyl, or acetyl, and
the substitution is performed by oxygen, nitrogen, sulfur, linear or branched C₁-C₆ alkyl, C₆-C₂₀ aryl, halogen, or a combination thereof).

According to an embodiment of the present disclosure, the compound represented by Chemical Formula 1 above may include a compound represented by Chemical Formula 2 below, but is not limited thereto:

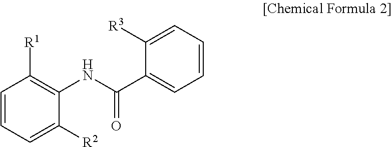

(wherein,
R¹ and R² are each independently hydrogen, substitutable linear or branched C₁-C₁₂ alkyl, substitutable C₃-C₁₂ cycloalkyl, or substitutable C₆-C₁₈ aryl,
R³ is carboxyl, hydroxyl, or acetyl, and
the substitution is performed by oxygen, nitrogen, sulfur, linear or branched C₁-C₆ alkyl, C₆-C₂₀ aryl, halogen, or a combination thereof).

According to an embodiment of the present disclosure, the compound represented by Chemical Formula 2 above may include the following compound, but is not limited thereto:

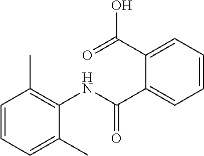 [SMILES: Cc1cccc(C)c1NC(=O)c1ccccc1C(=O)O]

According to an embodiment of the present disclosure, the compound represented by Chemical Formula 1 may inhibit senescence caused by an anti-cancer drug occurring in cancer-associated fibroblasts (CAFs) formed around cancer cells, but is limited thereto.

According to an embodiment of the present disclosure, the cancer may be selected from the group consisting of gastric cancer, esophageal cancer, pancreatic cancer, breast cancer, melanoma, thyroid cancer, liver cancer, glioblastoma, ovarian cancer, colon cancer, head and neck cancer, bladder cancer, renal cell cancer, metastatic cancer, prostate cancer, lung cancer and combinations thereof, but is not limited thereto.

According to an embodiment of the present disclosure, the cancer may be gastric cancer, but is not limited thereto.

According to an embodiment of the present disclosure, the anti-cancer drug may include at least one selected from the group consisting of 5-fluorouracil (5-FU), dacomitinib, osimertinib, cetuximab, Pyrotinib, Lcotinib, panitumumab, zalutumumab, Nimotuzumab, matuzumab, gefitinib, erlotinib, Lapatinib, neratinib, Vandetanib, necitumumab, afatinib, Taxol, Cisplatin, doxorubicin, paclitaxel, vincristine, topotecan, docetaxel, gleevec, carboplatin, daunorubicin, valrubicin, flutamide, gemcitabine, Etoposide and combinations thereof, but is not limited thereto.

According to a second aspect of the present disclosure, there is provided a pharmaceutical composition for preventing or treating cancer including the composition for inhibiting anti-cancer drug resistance according to the first aspect of the present disclosure.

According to an embodiment of the present disclosure, the cancer may be selected from the group consisting of gastric cancer, esophageal cancer, pancreatic cancer, breast cancer, melanoma, thyroid cancer, liver cancer, glioblastoma, ovarian cancer, colon cancer, head and neck cancer, bladder cancer, renal cell cancer, metastatic cancer, prostate cancer, lung cancer and combinations thereof, but is not limited thereto.

According to an embodiment of the present disclosure, the cancer may be gastric cancer, but is not limited thereto.

The above-mentioned technical solutions are merely exemplary and should not be construed as limiting the present disclosure. In addition to the above-described embodiments, additional embodiments may exist in the drawings and detailed description of the invention.

According to the present disclosure, the composition for inhibiting anti-cancer drug resistance may reduce anti-cancer drug resistance caused by senescent fibroblasts by inhibiting senescence of fibroblasts caused by chemotherapy. Specifically, when administering chemotherapy to fibroblasts extracted from gastric cancer, the fibroblasts change to a senescence-associated secretory phenotype (SASP), which appears in senescent cells, the senescent fibroblasts increase the anti-cancer drug resistance of gastric cancer. However, when treating the composition for inhibiting anti-cancer drug resistance according to the present disclosure, the senescent fibroblasts return to their original shapes, and as a result, anti-cancer drug resistance caused by the senescent fibroblasts may be reduced. In other words, the composition for inhibiting anti-cancer drug resistance according to the present disclosure has an effect of controlling the anti-cancer drug resistance of cancer cells by suppressing senescence of the tumor microenvironment.

However, effects obtainable herein are not limited to the effects described above, and other effects may be present.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a schematic diagram illustrating a mechanism of a cell senescence process;

FIG. 2 illustrates results of observing the shapes of fibroblasts under various conditions according to Experimental Example of the present disclosure;

FIG. 3 illustrates results of confirming the cell viability of SNU66, a gastric cancer cell, under various conditions according to Experimental Example of the present disclosure;

FIG. 4 illustrates results of observing the migration of SNU668, a gastric cancer cell, under various conditions according to Experimental Example of the present disclosure; and

FIG. 5 illustrates results of measuring the migration of SNU668, a gastric cancer cell, under various conditions according to Experimental Example of the present disclosure.

### DETAILED DESCRIPTION OF THE EMBODIMENT

Hereinafter, embodiments of the present disclosure will be described in detail so as to be easily implemented by those skilled in the art, with reference to the accompanying drawings. However, the present disclosure may be embodied in many different forms and are not limited to the embodiments to be described herein. In addition, parts not related with the description have been omitted in order to clearly describe the present disclosure in the drawings and throughout the present specification, like reference numerals designate like elements.

Throughout this specification, when a certain part is “connected” with the other part, it is meant that the certain part may be “directly connected” with the other part and “electrically connected” with the other part with another element interposed therebetween.

Throughout the present specification, it will be understood that when a certain member is located “on”, “above”, “at the top of”, “under”, “below”, and “at the bottom of” the other member, a certain member is in contact with the other member and another member may also be present between the two members.

Throughout the specification, when a part “comprises” a component, it will be understood to further include another component, not the exclusion of another component unless explicitly described to the contrary.

The terms “about”, “substantially”, and the like to be used in the present specification are used as a numerical value or a value close to the numerical value when inherent manufacturing and material tolerances are presented in the stated meaning, and used to prevent an unscrupulous infringer from unfairly using disclosed contents in which precise or absolute numerical values are mentioned to help in the understanding of the present disclosure. Throughout this specification, the term of “step to” or “step of” does not mean “step for”.

Throughout the present specification, the term “combinations thereof” included in the expression of the Markush form means one or more mixtures or combinations selected from the group consisting of components described in the expression of the Markush form, and means to include at least one selected from the group consisting of the components.

Throughout the present specification, “A and/or B” means “A or B, or A and B”.

Hereinafter, a composition for inhibiting anti-cancer drug resistance of the present disclosure will be described in detail with reference to embodiments, Examples, and drawings. However, the present disclosure is not limited to these embodiments, Examples and drawings.

As a technical means to achieve the technical problem, a first aspect of the present disclosure provides a composition for inhibiting anti-cancer drug resistance including a compound represented by Chemical Formula 1 below as an active ingredient:

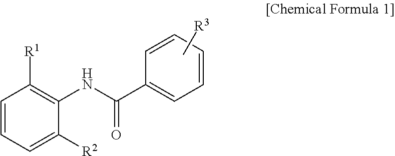

(wherein,
R¹ and R² are each independently hydrogen, substitutable linear or branched C₁-C₁₂ alkyl, substitutable C₃-C₁₂ cycloalkyl, or substitutable C₆-C₁₈ aryl,
R³ is carboxyl, hydroxyl, or acetyl, and
the substitution is performed by oxygen, nitrogen, sulfur, linear or branched C₁-C₆ alkyl, C₆-C₂₀ aryl, halogen, or a combination thereof).

The composition for inhibiting anti-cancer drug resistance according to the present disclosure may reduce anti-cancer drug resistance caused by senescent fibroblasts by inhibiting senescence of fibroblasts caused by chemotherapy. Specifically, when administering chemotherapy to fibroblasts extracted from gastric cancer, the fibroblasts change to a senescence-associated secretory phenotype (SASP), which appears in senescent cells, the senescent fibroblasts increase the anti-cancer drug resistance of gastric cancer. However, when treating the composition for inhibiting anti-cancer drug resistance according to the present disclosure, the senescent fibroblasts return to their original shapes, and as a result, anti-cancer drug resistance caused by the senescent fibroblasts may be reduced. In other words, the composition for inhibiting anti-cancer drug resistance according to the present disclosure has an effect of controlling the anti-cancer drug resistance of cancer cells by suppressing senescence of the tumor microenvironment.

According to an embodiment of the present disclosure, the compound represented by Chemical Formula 1 above may include a compound represented by Chemical Formula 2 below, but is not limited thereto:

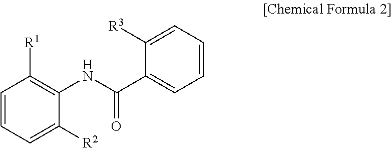

(wherein,
R¹ and R² are each independently hydrogen, substitutable linear or branched C₁-C₁₂ alkyl, substitutable C₃-C₁₂ cycloalkyl, or substitutable C₆-C₁₈ aryl,
R³ is carboxyl, hydroxyl, or acetyl, and
the substitution is performed by oxygen, nitrogen, sulfur, linear or branched C₁-C₆ alkyl, C₆-C₂₀ aryl, halogen, or a combination thereof).

According to an embodiment of the present disclosure, the compound represented by Chemical Formula 2 above may include the following compound, but is not limited thereto:

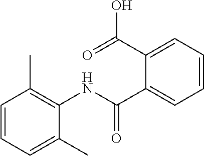 [SMILES: Cc1cccc(C)c1NC(=O)c1ccccc1C(=O)O]

The compound is a chemical drug approved by the FDA and is commonly used under the name ftaxilide. The compound is a drug generally used as an antifungal agent, and to date, there have been no reports of cases applied to the prevention or treatment of cancer using the compound.

According to an embodiment of the present disclosure, the compound represented by Chemical Formula 1 may inhibit senescence caused by an anti-cancer drug occurring in cancer-associated fibroblasts (CAFs) formed around cancer cells, but is limited thereto.

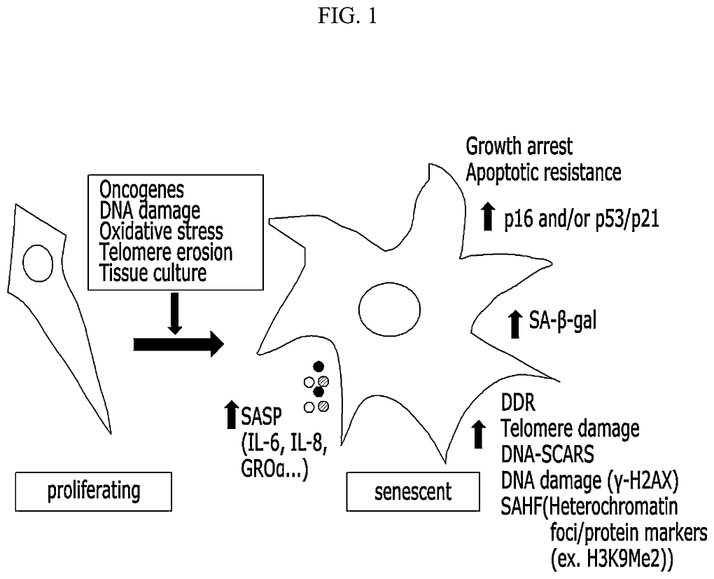

FIG. 1 is a schematic diagram illustrating a mechanism of a cell senescence process.

Referring to FIG. 1, generally, the senescence of cells may be caused by various factors such as oncogenes, DNA damage, oxidative stress, telomere erosion, and tissue culture. Despite the growth is inhibited, the senescent cells maintain a metabolic activity state and secrete various soluble or insoluble factors, and are known to change into the so-called senescence-associated secretory phenotype (SASP). Cells changed into SASP generally overexpress various secretion factors such as IL-6, IL-8, and CXCL1, which may affect a microenvironment within surrounding cells and tissues.

The present inventors confirmed the senescence occurring in gastric cancer-associated fibroblasts accumulated in gastric cancer tissues and an effect of senescent fibroblasts on gastric cancer cells, and confirmed that the compound represented by Chemical Formula 1 above inhibited the senescence of fibroblasts in a process of screening drugs for reducing the senescence of the senescent fibroblasts by treating various drugs that have not been previously studied or applied to cancer research on fibroblasts senescent by anti-cancer drugs.

According to an embodiment of the present disclosure, the cancer may be selected from the group consisting of gastric cancer, esophageal cancer, pancreatic cancer, breast cancer, melanoma, thyroid cancer, liver cancer, glioblastoma, ovarian cancer, colon cancer, head and neck cancer, bladder cancer, renal cell cancer, metastatic cancer, prostate cancer, lung cancer and combinations thereof, but is not limited thereto.

According to an embodiment of the present disclosure, the cancer may be gastric cancer, but is not limited thereto.

According to an embodiment of the present disclosure, the anti-cancer drug may include at least one selected from the group consisting of 5-fluorouracil (5-FU), dacomitinib, osimertinib, cetuximab, Pyrotinib, Lcotinib, panitumumab, zalutumumab, Nimotuzumab, matuzumab, gefitinib, erlotinib, Lapatinib, neratinib, Vandetanib, necitumumab, afatinib, Taxol, Cisplatin, doxorubicin, paclitaxel, vincristine, topotecan, docetaxel, gleevec, carboplatin, daunorubicin, valrubicin, flutamide, gemcitabine, Etoposide and combinations thereof, but is not limited thereto.

A second aspect of the present disclosure provides a pharmaceutical composition for preventing or treating cancer including the composition for inhibiting anti-cancer drug resistance according to the first aspect of the present disclosure.

With respect to the pharmaceutical composition for preventing or treating cancer according to the second aspect of the present disclosure, the detailed description of parts duplicated with the first aspect of the present disclosure has been omitted, but even if the description thereof has been omitted, the contents disclosed in the first aspect of the present disclosure may be equally applied to the second aspect of the present application.

The pharmaceutical composition for preventing or treating cancer according to the present disclosure includes the composition for inhibiting anti-cancer drug resistance according to the present disclosure to prevent the effect of the drug from being reduced due to anti-cancer drug resistance, thereby expecting more improved treatment effects.

According to an embodiment of the present disclosure, the cancer may be selected from the group consisting of gastric cancer, esophageal cancer, pancreatic cancer, breast cancer, melanoma, thyroid cancer, liver cancer, glioblastoma, ovarian cancer, colon cancer, head and neck cancer, bladder cancer, renal cell cancer, metastatic cancer, prostate cancer, lung cancer and combinations thereof, but is not limited thereto.

According to an embodiment of the present disclosure, the cancer may be gastric cancer, but is not limited thereto.

Hereinafter, the present disclosure will be described in more detail with reference to the following Examples, but the following Examples are only for illustrative purposes and are not intended to limit the scope of the present disclosure.

### [Example] Ftaxilide

Ftaxilide, a compound represented by Chemical Formula below, was used as a substance for inhibiting anti-cancer drug resistance.

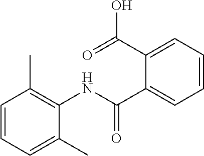 [SMILES: Cc1cccc(C)c1NC(=O)c1ccccc1C(=O)O]

### [Experimental Example 1] Confirmation of Inhibition of Senescence of Fibroblasts

An experiment was performed to confirm changes in shapes of fibroblasts by treating several fibroblasts CAF47, CAF51, CAF98, and CAF104 with an anti-cancer drug, 5-fluorouracil (5-FU), and then treating Example thereto.

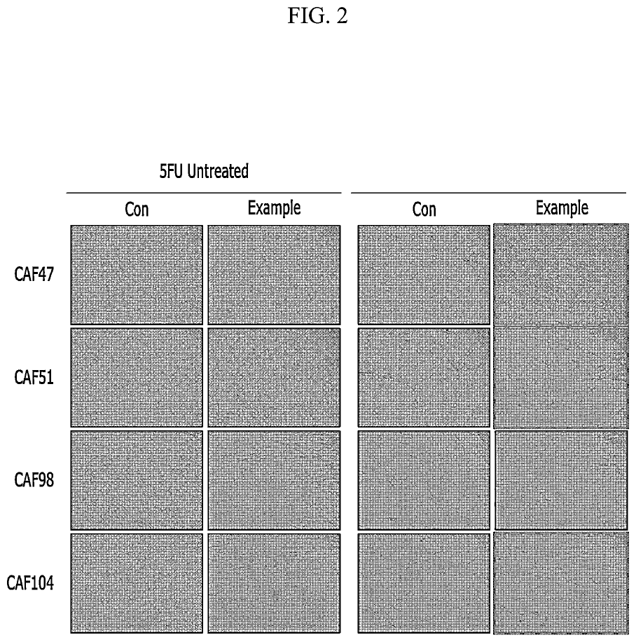

FIG. 2 illustrates results of observing the shapes of fibroblasts under various conditions according to Experimental Example of the present disclosure.

Referring to FIG. 2, when treating several fibroblasts with 5-FU, it was confirmed that as characteristics of senescent cells, the cell sizes increased and the cell growth rate slowed down, and when treating Example thereto, it was confirmed that the senescent fibroblasts returned to their original shapes.

### [Experimental Example 2] Comparison of Cell Viability of Gastric Cancer Cells

Gastric cancer cells, SNU668 cells, were cultured in conditioned media of fibroblasts, CAF98 and CAF104, respectively, and treated with Example to confirm changes in cell viability of cancer cells.

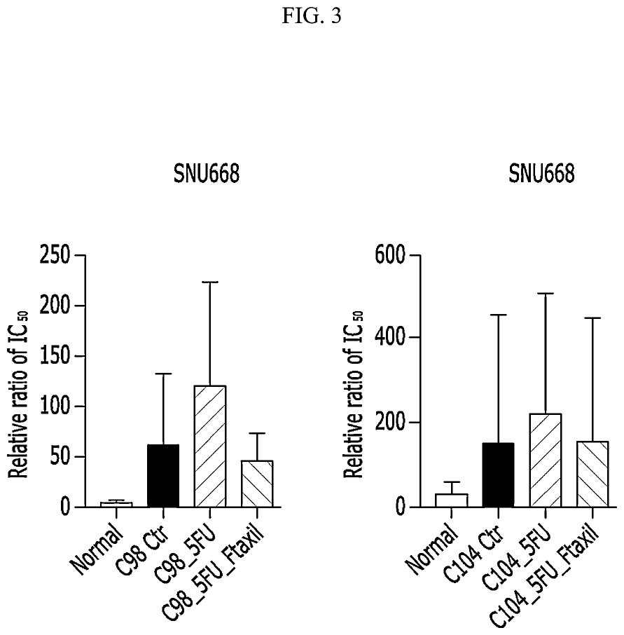

FIG. 3 illustrates results of confirming the cell viability of SNU66, a gastric cancer cell, under various conditions according to Experimental Example of the present disclosure.

Referring to FIG. 3, when gastric cancer cells SNU668 were cultured in the conditioned medium of CAF98 and CAF104 fibroblasts, it was confirmed that the cell viability increased (C98 Ctr and C104 Ctr), and when treated with 5-FU, it was confirmed that the cell viability of cancer cells further increased (C98_5FU and C104_5FU).

However, when further treated with Example, it was confirmed that the cell viability of cancer cells, which had been increased by the conditioned medium of fibroblasts or 5-FU, decreased again (C98_5FU_Ftaxil and C104_5FU_Ftaxil).

Through this, it could be seen that Example (ftaxilide) of the present disclosure inhibited the senescence of fibroblasts caused by anti-cancer drugs to inhibit the resistance of cancer cells to anti-cancer drugs, resulting in a decrease in the cell viability of cancer cells.

### [Experimental Example 3] Comparison of Migration of Gastric Cancer Cells

An experiment was performed to confirm changes in migration of gastric cancer cells when treating gastric cancer cells with a substance according to Example (ftaxilide).

SNU668 was co-cultured with 5-FU in various types of fibroblast condition media, and sequentially additionally treated with 5-FU and Example to confirm changes in the migration of gastric cancer cells.

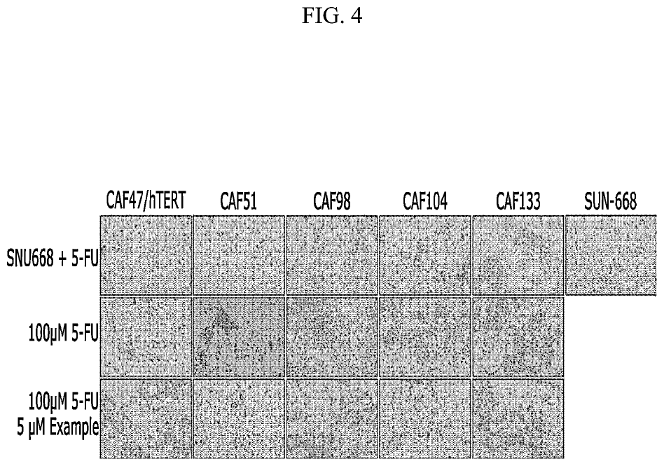

FIG. 4 illustrates results of observing the migration of SNU668, a gastric cancer cell, under various conditions according to Experimental Example of the present disclosure.

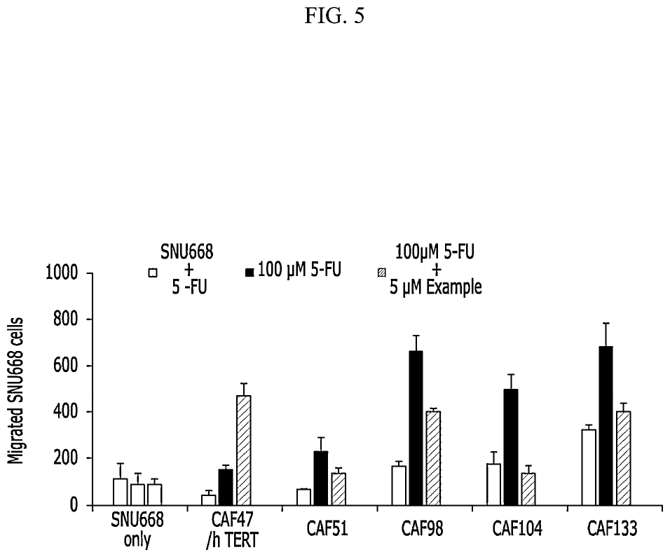

FIG. 5 illustrates results of measuring the migration of SNU668, a gastric cancer cell, under various conditions according to Experimental Example of the present disclosure.

Referring to FIGS. 4 and 5, it was confirmed that when treating gastric cancer cells SNU668 with 5-FU, the migration of gastric cancer cells increased (SNU668+5FU), and it was confirmed that when CAFs were senesced by treating 5-FU to fibroblasts (CAFs) and then co-cultured with gastric cancer cells SNU668, the migration of gastric cancer cells further increased (100 μM 5-FU).

However, when Example was treated with CAF, it was confirmed that the senescence of CAF was prevented and the migration of gastric cancer cells, which was increased by 5-FU or has been increased by CAF senesced by 5-FU, decreased again (100 μM 5-FU+5 μM Example).

Through this, it could be seen that Example (ftaxilide) of the present disclosure inhibited the senescence of fibroblasts caused by anti-cancer drugs to inhibit the resistance of cancer cells to anti-cancer drugs, resulting in a decrease in the cell viability of cancer cells.

The aforementioned description of the present disclosure is to be exemplified, and it will be understood by those skilled in the art that the present disclosure can be easily modified in other detailed forms without changing the technical spirit or required features of the present disclosure. Therefore, it should be appreciated that the embodiments described above are illustrative in all aspects and are not restricted. Therefore, it should be appreciated that the embodiments described above are illustrative in all aspects and are not restricted.

The scope of the present disclosure is represented by appended claims to be described below rather than the detailed description, and it is to be interpreted that the meaning and scope of the claims and all the changes or modified forms derived from the equivalents thereof come within the scope of the present disclosure.

1. A composition for inhibiting anti-cancer drug resistance comprising a compound represented by Chemical Formula 1 below as an active ingredient: 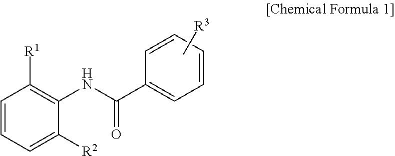
(wherein,
R¹ and R² are each independently hydrogen, substitutable linear or branched C₁-C₁₂ alkyl, substitutable C₃-C₁₂ cycloalkyl, or substitutable C₆-C₁₈ aryl,
R³ is carboxyl, hydroxyl, or acetyl, and
the substitution is performed by oxygen, nitrogen, sulfur, linear or branched C₁-C₆ alkyl, C₆-C₂₀ aryl, halogen, or a combination thereof).

2. The composition for inhibiting anti-cancer drug resistance of claim 1, wherein the compound represented by Chemical Formula 1 above comprises a compound represented by Chemical Formula 2 below: 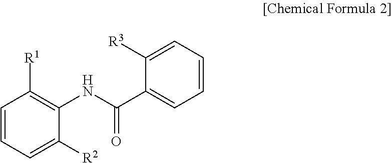
(wherein,
R¹ and R² are each independently hydrogen, substitutable linear or branched C₁-C₁₂ alkyl, substitutable C₃-C₁₂ cycloalkyl, or substitutable C₆-C₁₈ aryl,
R³ is carboxyl, hydroxyl, or acetyl, and
the substitution is performed by oxygen, nitrogen, sulfur, linear or branched C₁-C₆ alkyl, C₆-C₂₀ aryl, halogen, or a combination thereof).

3. The composition for inhibiting anti-cancer drug resistance of claim 2, wherein the compound represented by Chemical Formula 2 above comprises the following compound:

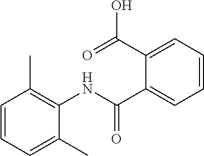 [SMILES: Cc1cccc(C)c1NC(=O)c1ccccc1C(=O)O]

4. The composition for inhibiting anti-cancer drug resistance of claim 1, wherein the compound represented by Chemical Formula 1 above inhibits senescence caused by an anti-cancer drug occurring in cancer-associated fibroblasts (CAFs) formed around cancer cells.

5. The composition for inhibiting anti-cancer drug resistance of claim 4, wherein the cancer is selected from the group consisting of gastric cancer, esophageal cancer, pancreatic cancer, breast cancer, melanoma, thyroid cancer, liver cancer, glioblastoma, ovarian cancer, colon cancer, head and neck cancer, bladder cancer, renal cell cancer, metastatic cancer, prostate cancer, lung cancer and combinations thereof.

6. The composition for inhibiting anti-cancer drug resistance of claim 5, wherein the cancer is gastric cancer.

7. The composition for inhibiting anti-cancer drug resistance of claim 1, wherein the anti-cancer drug is selected from the group consisting of 5-fluorouracil (5-FU), dacomitinib, osimertinib, cetuximab, Pyrotinib, Lcotinib, panitumumab, zalutumumab, Nimotuzumab, matuzumab, gefitinib, erlotinib, Lapatinib, neratinib, Vandetanib, necitumumab, afatinib, Taxol, Cisplatin, doxorubicin, paclitaxel, vincristine, topotecan, docetaxel, gleevec, carboplatin, daunorubicin, valrubicin, flutamide, gemcitabine, Etoposide and combinations thereof.

8. A pharmaceutical composition for preventing or treating cancer comprising the composition for inhibiting anti-cancer drug resistance according to claim 1.

9. The pharmaceutical composition for preventing or treating cancer of claim 8, wherein the cancer is selected from the group consisting of gastric cancer, esophageal cancer, pancreatic cancer, breast cancer, melanoma, thyroid cancer, liver cancer, glioblastoma, ovarian cancer, colon cancer, head and neck cancer, bladder cancer, renal cell cancer, metastatic cancer, prostate cancer, lung cancer and combinations thereof.

10. The pharmaceutical composition for preventing or treating cancer of claim 9, wherein the cancer is gastric cancer.
